(19) United States
(12) Patent Application Publication
(10) Pub. No.: US 2005/0265925 A1
(43) Pub. Date: Dec. 1, 2005

(54) Releasable linkage and compositions containing same

(76) Inventors: Samuel Zalipsky, Redwood City, CA (US); Paramjeet Subramony, Santa Clara, CA (US)

Correspondence Address: PERKINS COIE LLP, P.O. BOX 2168, MENLO PARK, CA, 94026, US

(21) Appl. No.: 11/110,272

(22) Filed: Apr. 20, 2005

Related U.S. Application Data
(60) Provisional application No. 60/564,565, filed on Apr. 21, 2004.

(51) Int. Cl. A61K 49/00; A61K 9/127

(52) U.S. Cl. 424/9.6; 424/450; 424/78.27

(57) ABSTRACT

Conjugates comprising a lipid or a hydrophilic polymer, such as polyethyleneglycol, linked to a ligand derived from an amine- or hydroxyl-containing compound, such as a drug or protein, are stable under conditions of storage, and are cleavable under mild thiolytic conditions to regenerate the amine- or hydroxyl-containing compound in its native form, without the formation of undesirable side products.

This application claims priority to U.S. Application Ser. No. 60/564,565, filed Apr. 21, 2004, which is hereby incorporated by reference in its entirety.

### FIELD OF THE INVENTION

The present invention relates to a conjugate comprising a lipid or a hydrophilic polymer, such as polyethyleneglycol, cleavably linked to a ligand derived from an amine- or hydroxyl-containing compound, such as a drug or protein. The conjugates are cleavable under mild thiolytic conditions to regenerate the amine- or hydroxyl-containing compound in its native form.

### REFERENCES

Blay, G. et al. A selective hydrolysis of aryl acetates. Synthesis 438 (1989).
Borchardt et al. Synthesis and evaluation of the physicochemical properties of esterase-sensitive cyclic prodrugs of opioid peptides using coumarinic acid and phenylpropionic acid linkers. J. Peptide Res. 53:370-382 (1999).
Ekrami, M. et al. Water-soluble fatty acid derivatives as acylating agents for reversible lipidization of polypeptides. FEBS Lett. 283-286 (1995).
Greenwald, R. B. et al. Coumarin and related aromatic based polymeric prodrugs. U.S. Pat. No. 6,214,330 (April 2001).
Harris, J. M. and Chess, R. B. Effect of pegylation on pharmaceuticals. Nat. Rev. Drug Discov. 2(3):214-21 (March 2003).
March, J. ADVANCED ORGANIC CHEMISTRY: REACTIONS, MECHANISM, AND STRUCTURE, Wiley-Interscience, 1992; p. 378.
Meth-Cohn, O. and Tarnowski, B. Thiocoumarins. Advances in Heterocyclic Chemistry 26:115-133 (1980).
Molineux, G. Pegylation: Engineering improved pharmaceuticals for enhanced therapy. Cancer Treat. Rev. 28 Suppl A: 13-6 April 2002).
Molineux, G. Pegylation: Engineering improved biopharmaceuticals for oncology. Pharmacotherapy 23(8 Pt 2):3S-8S (August 2003).
Owen, T. C. Amino alkanethiols from amino alcohols via aminoalkyl sulfates and thiazolidinethiones. J. Chem. Soc. C.: 1373-1376 (1967).
Panetta, J. A. and Rapoport, H. Synthesis of thiocoumarins from acrylic and propionic ortho esters and benzenethiols. J. Org. Chem. 47:2626-2628 (1982).
Quick, J. and Crelling, J. K. The acetyl function as a protecting group for phenols. J. Org. Chem. 43(1):155-6 (1978).
Roberts, M. J., Bentley, M. D., and Harris, J. M. Chemistry for peptide and protein PEGylation. Adv. Drug Deliv. Rev. 54(4):459-76 (Jun. 17, 2002).
Shen, W. C., Wang, J. and Shen, D. Reversible lipidization of polypeptides in drug delivery. Proceed. Intern. Sym. Control. Rel. Bioact. Mater. 24:202-203 (1997).
Zalipsky, S. Releasable linkage and compositions containing same. U.S. Pat. No. 6,342,244 (January 2002).
Zalipsky, S. et al. New detachable poly(ethylene glycol) conjugates: Cysteine-cleavable lipopolymers regenerating natural phospholipid, diacylphosphatidyl ethanolamine. Bioconjugate Chem. 10:703-707 (1999).
Zalipsky, S. et al. Polymer-protein conjugates as macromolecular prodrugs: Reversible PEGylation of proteins. Proc. Int'l. Symp. Control. Re. Bioact. Mater. 28: 73-74 (2001).
Zalipsky, S. et al. Reversible PEGylation: Thiolytic regeneration of active protein from its polymer conjugates; in PEPTIDES:THE WAVE OF THE FUTURE, M. Lebl and A. Houghten, eds., pp. 953-4, American Peptide Soc. (2001).

### BACKGROUND OF THE INVENTION

Hydrophilic polymers, such as polyethylene glycol (PEG), have been used for modification of various substrates, such as polypeptides, drugs and liposomes, in order to reduce immunogenicity of the substrate and/or to improve its blood circulation lifetime. For example, parenterally administered proteins can be immunogenic and may be rapidly degraded in vivo. Consequently, it can be difficult to achieve therapeutically useful blood levels of proteins in patients. Conjugation of PEG to proteins has been described as an approach to overcoming these difficulties. Davis et al., in U.S. Pat. No. 4,179,337, disclose conjugating PEG to proteins such as enzymes and insulin to form PEG-protein conjugates having less immunogenicity yet retaining a substantial proportion of physiological activity. Veronese et al. (Applied Biochem. and Biotech, 11: 141-152 (1985)) disclose activating polyethylene glycols with phenyl chloroformates to modify a ribonuclease and a superoxide dismutase. Katre et al., in U.S. Pat. Nos. 4,766,106 and 4,917,888, disclose solubilizing proteins by polymer conjugation. U.S. Pat. No. 4,902,502 (Nitecki et al.) and PCT Pubn. No. WO 90/13540 (Enzon, Inc.) describe conjugation of PEG and other polymers to recombinant proteins to reduce immunogenicity and increase half-life.

PEG has also been described for use in improving the blood circulation lifetime of liposomes (U.S. Pat. No. 5,103,556). The PEG is covalently attached to the polar head group of a lipid in order to mask or shield the liposomes from being recognized and removed by the reticuloendothelial system.

Because modification of a biologically active molecule, such as a protein, with a polymer often reduces the activity of the molecule, protein-polymer conjugates having cleavable linkages have been employed. Garman (U.S. Pat. No. 4,935,465) describes proteins modified with a water soluble polymer joined to the protein through a reversible linking group. Liposomes having releasable PEG chains have also been described, where the PEG chain is released from the liposome upon exposure to a suitable stimulus, such as a change in pH (WO 98/16201).

In some cases, release of the polymer from the liposome or molecule causes a change in structure of the molecule or lipid. These chemically modified structures can have unpredictable, potentially negative effects in vivo.

Conjugation strategies in which cleavage of a PEG-drug conjugate releases the drug are described in U.S. Pat. Nos. 6,342,244 and 6,214,330. The former describes cleavage of a dithiobenzyl moiety, with release of a side product such as thioquinonemethide. The latter describes cleavage of a hydrolytically labile aryl ether, with release of a side product such as coumarin.

In general, it is desirable to provide cleavable conjugates in which the linkage is stable under storage conditions but is cleavable in vivo to release the conjugated molecule in its original form, without the formation of undesirable side products.

### SUMMARY OF THE INVENTION

Accordingly, it is an object of the invention to provide a conjugate having a ligand covalently yet reversibly linked to a hydrophilic polymer. The ligand is derived from an amine- or hydroxy-containing compound. Upon cleavage of the linkage, the ligand in its native form is regenerated.

In one aspect, the invention includes a conjugate having the general structure I: 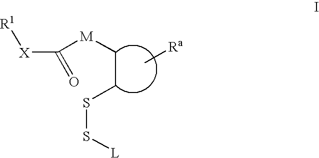
wherein

R¹X is an amine- or hydroxyl-containing ligand, such that X is oxygen, primary nitrogen or secondary nitrogen;

M is selected from cis —CR^{b}═CR^{c}—, —CR^{b}R^{d}—, and —CR^{b}R^{d}—CR^{c}R^{e}—, wherein each of R^{b}, R^{c}, R^{d}, and R^{e} is independently selected from H, methyl, substituted methyl, fluoro, and chloro, where methyl may be substituted with hydroxyl, fluoro, or chloro;

the D-shaped structure represents a five- or six-membered ring to which M and the disulfide group S—S are attached in a cis-1,2- or ortho orientation;

R^{a} represents hydrogen or one or more substituents on the ring selected from R, OR, C(O)OH, C(O)OR, OC(O)OR, C(O)NR₂, OC(O)NR₂, cyano, nitro, halogen, and a further fused ring, where R is C₁-C₆ hydrocarbyl, which may be further substituted with halogen; and

L is a linear or branched C₁-C₆ alkyl group, which may be further substituted with aryl or aralkyl;

wherein L and R^{a} may together form a ring;

and wherein the conjugate further comprises, attached to L, to R^{a}, or to the five- or six-membered ring, a lipid or a hydrophilic polymer.

The conjugate typically comprises a hydrophilic polymer attached to L or to R^{a}. In selected embodiments, L and R^{a} do not form a ring.

The hydrophilic polymer may be, for example, polyvinylpyrrolidone, polyvinylmethylether, polymethyloxazoline, polyethyloxazoline, poly(hydroxypropyl) oxazoline, poly(hydroxypropyl)methacrylamide, polymethacrylamide, polydimethyl acrylamide, poly(hydroxypropyl)methacrylate, poly(hydroxyethyl)acrylate, hydroxymethyl cellulose, hydroxyethylcellulose, polyethylene glycol, polypropylene glycol, polyaspartamide, and copolymers thereof; a preferred hydrophilic polymer is a polyether, such as polyethylene glycol.

Preferably, the five- or six-membered ring is an aromatic ring, more preferably a benzene ring. In one embodiment, where M is cis —CR^{b}═CR^{c}—, the conjugate has the structure Ia:

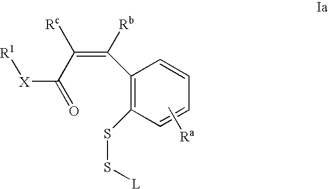

In this embodiment, each of R^{b} and R^{c} is preferably hydrogen. Preferably, the hydrophilic polymer is attached to L and not to R^{a}.

R^{a} may be, for example, hydrogen or a single substituent selected from R, OR, C(O)OH, C(O)OR, OC(O)OR, C(O)NR₂, OC(O)NR₂, cyano, nitro, fluoro, chloro, where R is C₁-C₆ hydrocarbyl, which may be further substituted with halogen. Preferably, R^{a} is hydrogen or a single substituent selected from R, OR, C(O)OR, C(O)OH, cyano, nitro, fluoro, and chloro, where R is methyl or ethyl. In selected embodiments, R^{a} is hydrogen.

Preferably, L has the structure —CR³R⁴—CR⁵R⁶—, such that —CR³R⁴ is attached to the disulfide group, where R³ and R⁴ are independently selected from H, alkyl, aryl, and aralkyl, and R⁵ and R⁶ are independently selected from H and methyl. Preferably, each of R³ and R⁴ is independently selected from hydrogen, methyl, ethyl, and propyl. More preferably, R⁴ is H and R³ is selected from the group consisting of hydrogen, methyl, ethyl, and propyl. In selected embodiments, R⁴ is H and R³ is selected from the group consisting of CH₃, C₂H₅ and C₃H₈.

In one embodiment of the structure I above, L and R^{a} are attached to the five- or six-membered ring in a cis-1,2- or ortho orientation, and L and R^{a} together form a further five- to seven-membered ring. In such embodiments, a hydrophilic polymer attached to the five- or six-membered ring (i.e. the “D-shaped structure”, preferably a benzene ring), or it may be attached to the further five- to seven-membered ring formed by L and R^{a}.

The ligand represented by R¹X is typically a lipid or a biologically active compound. In selected embodiments, the ligand is an amine-containing ligand, which may be, for example, a polypeptide, an amine-containing drug, or an amine-containing lipid. The amine-containing lipid is preferably a phospholipid having a double hydrocarbon tail group. When the ligand is derived from a polypeptide, the polypeptide may be, for example, an enzyme or a cytokine.

In a related aspect, the invention provides a conjugate obtainable by reaction of an amine- or hydroxyl-containing molecule with a compound having the structure II: 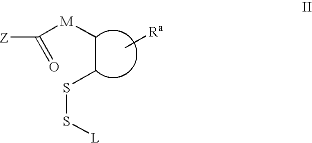
wherein

Z is a leaving group displaceable by a hydroxyl or amino group;

M is selected from cis —CR^{b}═CR^{c}—, —CR^{b}R^{d—}, and —CR^{b}R^{d}—CR^{c}R^{e}—, wherein each of R^{b}, R^{c}, R^{d}, and R^{e} is independently selected from H, methyl, substituted methyl, fluoro, and chloro, where methyl may be substituted with hydroxyl, fluoro, or chloro;

the D-shaped structure represents a five- or six-membered ring to which M and the disulfide group S—S are attached in a cis-1,2- or ortho orientation;

R^{a} represents hydrogen or one or more substituents on the ring selected from R, OR, C(O)OH, C(O)OR, OC(O)OR, C(O)NR₂, OC(O)NR₂, cyano, nitro, halogen, and a further fused ring, where R is C₁-C₆ hydrocarbyl, which may be further substituted with halogen; and

L is a linear or branched C₁-C₆ alkyl group, which may be further substituted with aryl or aralkyl;

wherein L and R^{a} may together form a ring;

and wherein the compound further comprises, attached to L, to R^{a}, or to the five- or six-membered ring, a lipid or a hydrophilic polymer.

Preferred embodiments of structure II, i.e. with respect to the variables M, L, and Ra, and the lipid or hydrophilic polymer, correspond to those described for structure I above. For example, in one embodiment, the compound has the structure IIa, where the five- or six-membered ring is a benzene ring, and M is cis —CR^{b}═CR^{c}—.

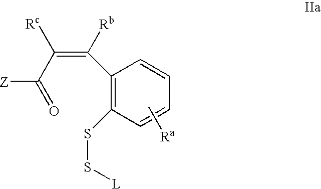

The leaving group Z is preferably selected from the group consisting of chloride, para-nitrophenol, ortho-nitrophenol, N-hydroxytetrahydrophthalimide, N-hydroxysuccinimide, N-hydroxyglutarimide, N-hydroxynorbornene-2,3-dicarboxyimide, 1-hydroxybenzotriazole, 3-hydroxypyridine, 4-hydroxypyridine, 2-hydroxypyridine, 1-hydroxy-6-trifluoromethylbenzotriazole, imidazole, triazole, N-methylimidazole, pentafluorophenol, trifluorophenol, and trichlorophenol.

In another aspect, the invention provides a method for administering an amine- or hydroxyl-containing molecule R²XH to the bloodstream, by administering to the bloodstream a conjugate having the structure I, as described above, whereby the molecule R²XH is released from the conjugate via an in vivo thiolytic cleavage reaction of the conjugate. Preferred embodiments of the conjugate are as described above. The method may further comprise monitoring the release of the molecule via detection of a fluorescent moiety released by the cleavage reaction.

In a further aspect, the invention provides a liposome having a surface coating of hydrophilic polymer chains, and comprising a lipid-polymer conjugate having the structure I as described above, where R¹X represents an amine- or hydroxyl-containing lipid, preferably a phospholipid. Preferred embodiments of other variables within the structure I are as described above. The liposome may include an entrapped therapeutic agent. In a related aspect, the invention provides a liposomal composition comprising such a liposome, and further comprising vesicle-forming lipids stably linked to a hydrophilic polymer. Preferably, the total mole percent of lipids linked to a hydrophilic polymer is between 1% and about 20%. In a preferred embodiment of the liposomal composition, hydrophilic polymers stably linked to vesicle-forming lipids are shorter than those contained in conjugates of structure I.

Also provided are compositions containing a conjugate as described above and a pharmaceutically-acceptable carrier, such as saline, buffer or the like.

These and other objects and features of the invention will be more fully appreciated when the following detailed description of the invention is read in conjunction with the accompanying drawings.

### BRIEF DESCRIPTION OF THE DRAWINGS

FIG. 1 shows a conjugate in which dithiocinnamyl (DTC) links a methoxy-polyethylene glycol (mPEG) moiety and an amine-containing ligand, in accordance with one embodiment of the invention;

FIG. 2 illustrates a synthetic reaction scheme for synthesis of mPEG-DTC-NHS ester conjugate;

FIG. 3 shows thiolytic cleavage of the mPEG-DTC-protein conjugate of FIG. 1, and the resulting products; and

FIG. 4 shows thiolytic cleavage of another conjugate of the invention, and the resulting products.

### DETAILED DESCRIPTION OF THE INVENTION

I. Definitions

A “polypeptide”, as used herein, is a polymer of amino acids, without limitation as to a specific length. Thus, for example, the terms peptide, oligopeptide, protein, and enzyme are included within the definition of polypeptide. This term also includes post-expression modifications of the polypeptide, for example, glycosylations, acetylations, phosphorylations, and the like.

A “hydrophilic polymer”, as used herein, refers to a polymer having moieties soluble in water, which lend to the polymer some degree of water solubility at room temperature. Exemplary hydrophilic polymers include polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropyl methacrylate, polyhydroxyethylacrylate, hydroxymethylcellulose, hydroxyethylcellulose, polyethyleneglycol, polyaspartamide, copolymers of the above-recited polymers, and polyethyleneoxide-polypropylene oxide copolymers. Properties and reactions of many of these polymers are described in U.S. Pat. Nos. 5,395,619 and 5,631,018.

A “polymer comprising a reactive functional group” or a “polymer comprising a linkage for attachment” refers to a polymer that has been modified, typically (but not necessarily) at a terminal end moiety, for reaction with another compound to form a covalent linkage. Reaction schemes effective to functionalize a polymer to have such a reactive functional group are readily determined by those of skill in the art and/or have been disclosed, for example in U.S. Pat. No. 5,613,018; in Zalipsky et al., Eur. Polymer. J. 19(12): 1177-1183 (1983); or in Zalipsky et al., Bioconj. Chem. 4(4):296-299 (1993).

“Alkyl”, as used herein, refers to a group derived from an alkane by removal of a hydrogen atom from any carbon atom, and having the formula CₙH₂ₙ₊₁. The groups derived by removal of a hydrogen atom from a terminal carbon atom of unbranched alkanes form a subclass of normal alkyl (n-alkyl) groups: H[CH₂]ₙ. The groups RCH₂—, R₂CH— (R not equal to H), and R₃C— (R not equal to H) represent primary, secondary and tertiary alkyl groups respectively.

“Lower alkyl” refers to alkyl groups having 1-6, and more preferably 1-4, carbon atoms.

“Hydrocarbyl” encompasses groups consisting of carbon and hydrogen; i.e. alkyl, alkenyl, alkynyl, cycloalkyl, cycloalkenyl, and non-heterocyclic aryl.

“Aryl” refers to a substituted or unsubstituted monovalent aromatic radical having a single ring (e.g., phenyl), two condensed rings (e.g., naphthyl) or three condensed rings (e.g. anthracyl or phenanthryl). This term generally includes heteroaryl groups, which are aromatic ring groups having one or more nitrogen, oxygen, or sulfur atoms in the ring, such as furyl, pyrrole, pyridyl, and indole. By “substituted” is meant that one or more ring hydrogens in the aryl group is replaced with a halide such as fluorine, chlorine, or bromine; with a lower alkyl group containing one or two carbon atoms; or with nitro, amino, methylamino, dimethylamino, methoxy, halomethoxy, halomethyl, or haloethyl.

“Aralkyl” refers to a lower alkyl (preferably C₁-C₄, more preferably C₁-C₂) substituent which is further substituted with an aryl group; examples are benzyl and phenethyl.

An “aliphatic disulfide” linkage or bond refers to a linkage of the form R′—S—S—R″, where each of R′ and R″ is a linear or branched alkyl chain, which may be further substituted.

“A “stable” linkage, as used herein, refers to a linkage comprising functional groups which are appreciably more stable in vivo than the disulfide linkages described herein. Examples include, but are not limited to, amides, ethers, and amines.

“Vesicle-forming lipids” refers to amphipathic lipids which have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group moiety oriented toward the exterior, polar surface of the membrane. Such vesicle-forming lipids typically include one or two hydrophobic acyl hydrocarbon chains or a steroid group and may contain a chemically reactive group, such as an amine, acid, ester, aldehyde or alcohol, at the polar head group. Examples include phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation. Other vesicle-forming lipids include glycolipids, such as cerebrosides and gangliosides, and sterols, such as cholesterol.

II. Storage Stable In Vivo Cleavable Conjugates of the Invention

A. Structure

The invention provides conjugates in which a molecule, such as a biologically active molecule or a lipid constituent of a liposome, is linked to a further moiety via an in vivo cleavable linkage. The attached moiety is typically provided to enhance the pharmacological properties of the molecule; for example, to reduce immunogenicity and/or to enhance solubility or circulation time within the body after administration. The linkage is then cleaved in vivo to release the molecule in its original, biologically active form.

Frequently, the conjugate comprises a protein or other amine- or hydroxyl-containing molecule linked to polyethylene glycol (PEG). However, conjugates can be formed between virtually any two molecules containing suitable functional groups, for example, lipid-protein or lipid-drug conjugates, for enhanced gastrointestinal and BBB transport, lipid-polymer conjugates for use in surface-modified liposomes, etc.

In one aspect, the invention provides a disulfide-containing conjugate having the general structure I, which is linked to a lipid or polymer, as described below:

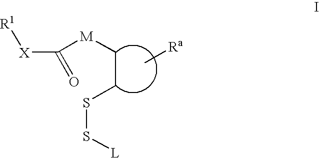

In the structure I, R¹X represents an amine- or hydroxyl-containing ligand, such that X is oxygen, primary nitrogen or secondary nitrogen, derived from a molecule (e.g. R¹XH or R¹XH₂) to be released following cleavage of the conjugate. The molecule may be a biologically active compound, such as a protein, polypeptide or small molecule drug compound. Alternatively, the ligand may be derived from an amine-containing lipid, typically a phospholipid, e.g. a phosphatidyl ethanolamine having a double hydrocarbon tail group.

The “D” -shaped structure in formula I represents a five- or six-membered ring. The ring may be saturated, e.g. cyclohexane, cyclopentane, or heterocycles such as tetrahydrofuran, tetrahydropyran, piperidine, pyrrolidine, or morpholine. Alternatively, the ring may be unsaturated, e.g. cyclohexene. Preferably, the ring is an aromatic ring, e.g. benzene, naphthalene, or anthracene, and is more preferably benzene. Also included are heteroaromatic rings, where one or more ring atoms (excluding those to which the groups S—S and M are attached) are replaced with nitrogen, oxygen, or sulfur. Preferred monocyclic systems include pyridine, pyrimidine, 2,4-imidazole, -thiazole, and -oxazole, and 2,5-pyrrole, -furan, and -thiophene. Preferably, the ring is a carbocyclic ring. Most preferably, the ring is a benzene ring.

The group M and the disulfide group (—S—S—) are attached to the five- or six-membered ring in a cis-1,2- or ortho orientation. M itself is selected from cis —CR^{b}═CR^{c}—, —CR^{b}R^{d}—, and —CR^{b}R^{d}—CR^{c}R^{e}—, where each of R^{b}, R^{c}, R^{d}, and R^{e} is independently selected from H, methyl, substituted methyl, fluoro, and chloro, where methyl may be substituted with hydroxyl, fluoro, or chloro. Preferably, each of R^{b}, R^{c}, R^{d}, and R^{e} is independently selected from H, and methyl; in one embodiment, each of R^{b}, R^{c}, R^{d}, and R^{e} is H.

In a preferred embodiment, the ring is a benzene ring and M is cis —CR^{b}═CR^{c}—, giving the structure Ia:

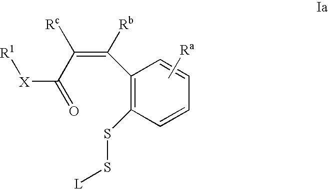

With reference to structures I and Ia, R^{a} represents hydrogen or one or more substituents on the five- or six-membered ring, selected from R, OR, C(O)OH, C(O)OR, OC(O)OR, C(O)NR₂, OC(O)NR₂, cyano, nitro, halogen, and a further fused ring, where R is C₁-C₆ hydrocarbyl, preferably C₁-C₄ hydrocarbyl, which may be further substituted with halogen. Halogen is preferably fluoro or chloro, and R preferably includes zero to two halogen substituents.

Preferably, a further fused ring, when present, contains five to seven ring atoms, preferably five or six ring atoms. Any stable fused ring system may be included. Examples include, but are not limited to, naphthalene, 2,6- or 2,7-benzimidazole, -benzothiazole, and -benzoxazole, 2,4- or 2,6-indole, quinoline, and analogs in which one or more non-fusing carbon atoms on a 5-ring or 6-ring are replaced with nitrogen.

In selected embodiments, R^{a} is hydrogen or a single substituent selected from R, OR, C(O)OH, C(O)OR, OC(O)OR, C(O)NR₂, OC(O)NR₂, cyano, nitro, and halogen, as described above, where R is as defined above. In further embodiments, R^{a} is hydrogen or a single substituent selected from R, OR, C(O)OR, cyano, nitro, carboxyl, fluoro, and chloro, where R is methyl or ethyl. In one embodiment, R^{a} is hydrogen.

L represents a linear or branched C₁-C₆ alkyl group, which may be further substituted with aryl. Preferably, L has the structure —CR³R⁴—CR⁵R⁶—, such that —CR³R⁴ is attached to the disulfide group, where R³ and R⁴ are independently selected from H, alkyl, aryl, and aralkyl, and R⁵ and R⁶ are independently selected from H and methyl.

In structure I, L and R^{a} may together form a ring, preferably a five- to seven-membered ring. In this case, R^{a} and the disulfide group (—S—S—) are preferably attached to the five- or six-membered ring (the benzene ring in Ia) in a cis-1,2- or ortho orientation.

The conjugate further includes, attached to L, to R^{a}, or to the five- or six-membered ring in structure I, a lipid or a hydrophilic polymer; that is, the moiety to which the ligand R¹X is to be conjugated. Examples of possible sites of attachment, where the lipid or hydrophilic polymer is designated R², are given in the structures (i-iv) below. For example, the lipid or hydrophilic polymer may be attached to the terminus of L, as in structure (i) below, or it may be attached to the five- or six-membered ring, either directly (e.g. structure (ii)) or via the substituent R^{a} (e.g. structure (iv)). Also included are embodiments in which R^{a} and L themselves are linked to form a ring (structure (iii)), and R² is attached to this ring (typically by virtue of attachment to either R^{a} or L).

In selected embodiments (e.g. (i) and (ii)), R^{a} and L do not form a ring. In further embodiments, R² is attached to a terminus of L (structure (i) below).

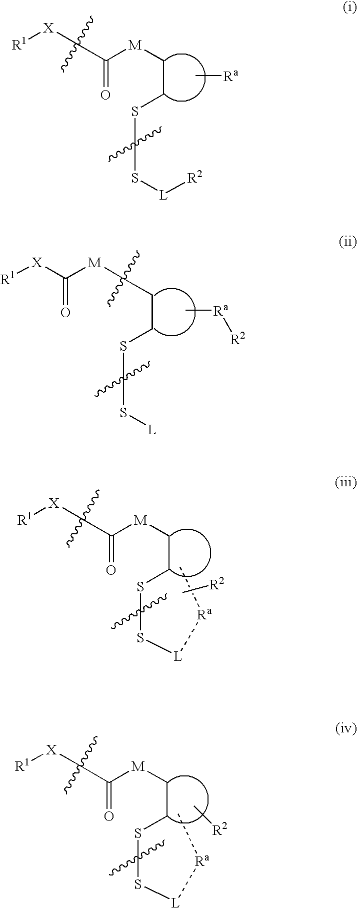

Whether or not L is linked to R^{a} to form a ring determines whether the conjugate generates two or three fragments (one of which the molecule R¹XH or R¹XH₂, in its native form) upon cleavage. As shown by the structures above, where wavy lines represent eventual cleavage locations, structures (i-ii) produce three fragments upon cleavage, and conjugates (iii-iv) generate two fragments upon cleavage. This aspect of the invention will be described in more detail below.

As also discussed further below, variation of the substitution of L at the position adjacent the disulfide group (e.g. variation of R³ and/or R⁴, when L=—CR³R⁴—CR⁵R⁶—) can be used to modulate the rate of cleavage of the conjugate. For example, to achieve a faster rate of cleavage, R³ and R³ are hydrogen. A slower rate of cleavage is achieved by sterically hindering the disulfide, by selecting an alkyl, aralkyl or aryl group for one or both of R³ and R⁴. Preferably, R³ and R⁴ are independently selected from hydrogen and lower (C₁ to C₆) alkyl. In selected embodiments, each of R³ and R⁴ is independently selected from hydrogen, methyl, ethyl, and propyl.

The lipid or hydrophilic polymer is typically linked to the structure I via a stable linker group, such as an amide, ester, carbamate, or sulfur analog thereof, where amides and carbamates are preferred. Methods of conjugation via such linker groups are well known in the art. For example, methods for linking PEG to various moieties via such groups are described, for example, in Zalipsky et al., 1999, 2001; Zalipsky, 2002; Roberts et al., 2002; Molineux, 2002, 2003; Harris et al., 2003; and other sources.

The hydrophilic polymer or lipid may also include a targeting moiety, typically attached to its free terminus. Such targeting moieties include those described in co-owned U.S. Pat. No. 6,660,525, which is incorporated herein by reference. Non-limiting examples of targeting moieties include antibodies, folate, for targeting epithelial carcinomas and bone marrow stem cells; pyridoxyl phosphate, galactose, for targeting liver hepatocytes; apolipoproteins, for targeting liver hepatocytes and vascular endothelial cells; transferrin, for targeting brain endothelial cells; VEGF, for targeting tumor epithelial cells; VCAM-1 or ICAM-1, for targeting vascular endothelial cells; Mac-1, for targeting neutrophils and leukocytes; HIV GP 120/41 or HIV GP 120 C4 domain peptomers, for targeting CD4+ lymphocytes; fibronectin, for targeting activated platelets; and osteopontin, for targeting endothelial cells and smooth muscle cells in atherosclerotic plaques.

For some ligands, such as polypeptide ligands, which have a variety of functional side groups, multiple polymers R² can be conjugated to the ligand. They may be conjugated via multiple structures I shown above, alone or in combination with a linkage which is more stable in vivo. The selection of the molecular weight of the polymers may depend on the number of polymer chains attached to the ligand, where a larger molecular weight polymer is often selected when the number of attached polymer chains is small, and vice versa.

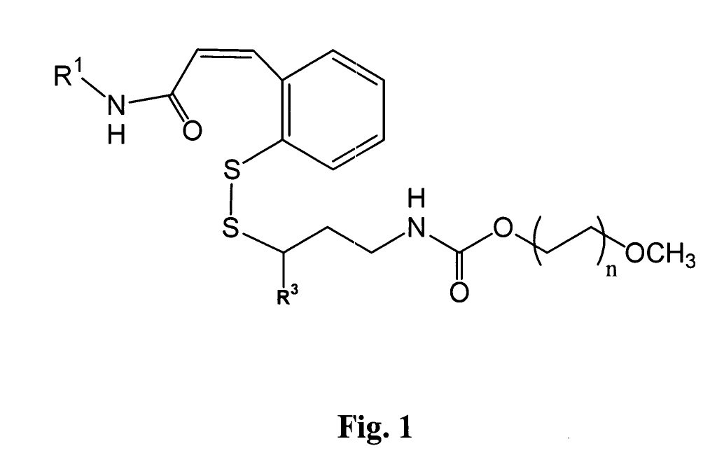

FIG. 1 shows the structure of an exemplary conjugate in accordance with the invention. The conjugate is an embodiment of structure Ia above, where each of R^{a}—R^{c} is hydrogen. Accordingly, the conjugate is based on a dithiocinnamate (DTC) structure.

R² in this conjugate is the hydrophilic polymer methoxy-polyethylene glycol (mPEG), which may be represented by the formula CH₃O(CH₂CH₂O)ₙ, where n is preferably about 10 to about 2300, which corresponds to molecular weights of about 440 Daltons to about 100,000 Daltons. The selection of the molecular weight of the polymer depends to some extent on the selection of the attached ligand. In embodiments where the ligand is derived from an amine-containing lipid, for use in a liposome, a preferred range of PEG molecular weight is from about 750 to about 10,000 Daltons, more preferably from about 2,000 to about 5,000 Daltons. In embodiments where the ligand is derived from an amine-containing polypeptide, a preferred range of PEG molecular weight is from about 2,000 to about 40,000 Daltons, more preferably from about 2,000 to about 20,000 Daltons. It will be appreciated that R2 can be selected from a variety of hydrophilic polymers, as well as lipids. Exemplary polymers are recited above.

In this conjugate, L is —CR³R⁴—CR⁵R⁶—, where R⁴—R⁶ are hydrogen and R³ is variable. As described above, R³ may be hydrogen, alkyl, aryl, or aralkyl. XR¹ in this conjugate is a primary amine-containing molecule, e.g. a drug or protein. The mPEG is attached to the terminus of L via a urethane (carbamate) group.

B. Synthesis

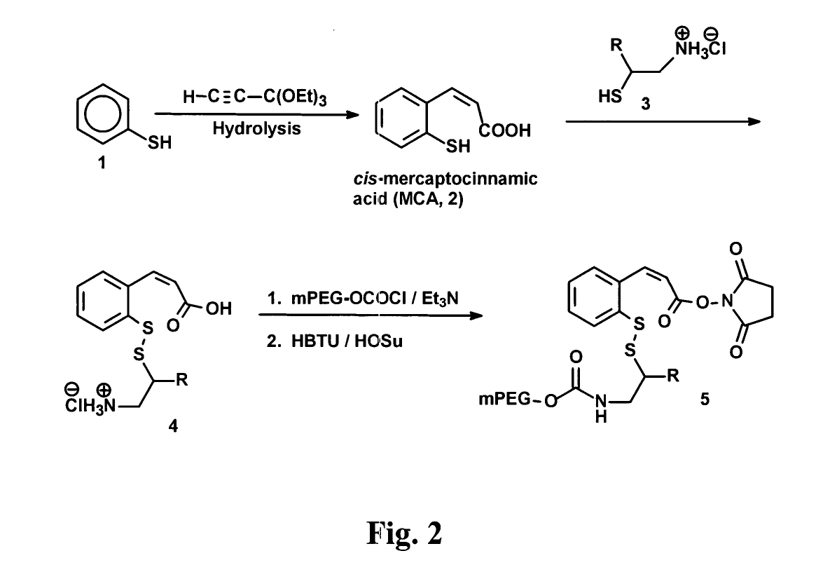

FIG. 2 illustrates an exemplary method, also described in Examples 1-6 below, for synthesis of an exemplary PEG-protein conjugate. The scheme could be readily modified, e.g. by substitution of a different molecule R¹ or polymer R², or by varying substitution on linker groups and/or rings, by one skilled in the art of organic synthesis and bioconjugation chemistry.

As noted above, in a preferred embodiment, the ring to which the disulfide is attached is a benzene ring, and M comprises a cis-olefin. cis-Mercaptocinnamic acids, in accordance with this embodiment, can be synthesized by addition of an orthoester-substituted alkyne to a thiophenol, according to a published procedure (Panetta and Rapoport, 1982). This procedure was used to prepare cis-mercaptocinnamic acid (2) from thiophenol (1) and triethyl orthopropiolate, as described in Example 1 below.

Attachment of the linking group L in FIG. 2 is accomplished by reaction with an alkanethiol having at its distal terminus a functional group useful for further conjugation, in this case an amino group. The reagent illustrated, 2-mercaptopropylamine hydrochloride (3, R═CH₃), can be prepared from the corresponding amino alcohol, according to the method of Owen, 1967. Analogous aminoalkylthiol derivatives with various R groups can be prepared in a similar fashion.

The mixed disulfide (4) can be formed via reaction of the aminoalkanethiol with an activating agent such as diethyl azidocarboxylate, followed by reaction with the aromatic thiol, e.g. according to the method of Mukaiyama et al., Tetrahedron Letters 56:5907-5908 (1968). Alternatively, methoxycarbonylsulfenyl chloride can be used to form an activated disulfide with the aminoalkanethiol, as described in S. J. Brois et al., J. Am. Chem. Soc. 92:7629-31 (1970). The activated disulfide can be reacted with the aromatic thiol to form disulfide (4) (see Zalipsky et al., 1999).

The terminal amino group of L is then used for attachment of the R² moiety, in this case for attachment of PEG via a urethane (carbamate) linkage. This can be accomplished by reaction with mPEG-chloroformate, according to various published protocols (see e.g. Zalipsky and Menon-Rudolph, in “Poly(ethylene glycol): Chemistry and Biological Applications”, J. M. Harris & S. Zalipsky, eds., Amer. Chem. Soc., Washington D.C., pp. 318-341 (1997)). The polymeric chloroformate can be generated by phosgenation of an anhydrous mPEG-OH solution, according to Zalipsky et al., Biotechnol. Appl. Biochem. 15:100 (1992). Alternatively, the linkage can be formed by reaction of mPEG-succinimidyl carbonate and the terminal amine, also according to known methods (see e.g. H. C. Chiu et al., Bioconjugate Chem. 4:290-295 (1993); Zalipsky et al., 1992; and Zalipsky and Menon-Rudolph, 1997; both cited above).

The protein (or other molecule to be conjugated, e.g. an amine- or hydroxyl-containing drug) is then conjugated to the free carboxyl group according to standard methods. For example, the acid can be converted to its N-hydroxy succinimide ester (5) using carbodiimide-mediated esterification procedure (see e.g. G. W. Anderson et al., J. Amer. Chem. Soc. 86:1839 (1964) ). Alternatively, this can be achieved with the reagent O-(N-succinimidyl)-N,N,N′,N′-tetramethyluronium hexafluorophosphate (R. Knorr et al., Tetrahedron Lett. 30(15): 1927-30 (1989); M. Wilchek et al., Bioconjugate Chem. 5:491 (1994)).

There are a number of general protocols for reacting amino groups of proteins with an N-hydroxysuccinimide ester. For representative procedures see e.g. Zalipsky et al., Biotechnol. Appl. Biochem. 15:100 (1992) or H. C. Chiu et al., Bioconjugate Chem. 4:290 (1993).

Depending on various parameters of such reactions, e.g. the amount of NHS reagent, the number of amino groups on the protein, the pH of the reaction buffer, the temperature, and the duration of the reaction, one can obtain a range of protein-polymer conjugate species having varying degrees of PEGylation. If necessary, conjugate mixtures of the general formula (mPEG)ₙ-protein can be fractionated by various chromatographic techniques. It is often possible to isolate 1:1 conjugates (i.e. where n=1), e.g. by ion-exchange chromatography.

Pertinent to the above syntheses, the invention also includes a composition comprising a conjugate obtainable by reaction of an amine-, hydroxy- or carboxyl-containing compound with a compound having the general structural formula II: 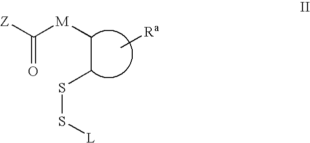
where M, R^{a}, and L are as described above, Z is a leaving group, and the compound further includes, attached to L, to R^{a}, or to the five- or six-membered ring represented by the D-shaped structure, as described above, a lipid or a hydrophilic polymer.

The leaving group Z is displaceable by reaction with an amine- or hydroxy-containing ligand compound, such as DSPE, a polypeptide, or an amine-containing drug. The leaving group is selected according to the reactivity of the displacing group in the ligand compound. Suitable leaving groups include chloride, p-nitrophenol, o-nitrophenol, N-hydroxy tetrahydrophthalimide, N-hydroxysuccinimide, N-hydroxy-glutarimide, N-hydroxy norbornene-2,3-dicarboxyimide, 1-hydroxybenzotriazole, 3-hydroxypyridine, 4-hydroxypyridine, 2-hydroxypyridine, 1-hydroxy-6-trifluoromethylbenzotriazole, imidazole, triazole, N-methyl-imidazole, pentafluorophenol, trifluorophenol, and trichlorophenol. Typically, such reaction forms an ester or amide linkage to the ligand.

C. Cleavage of the Conjugates

As noted above, cleavage of the conjugates is initiated by cleavage of the disulfide linkage. This occurs in vivo by a thiolytic mechanism, initiated by endogenous reagents such as cysteine or glutathione. The rate of cleavage can be modulated by varying the structure of the linker adjacent the disulfide group, and cleavage rates can be evaluated in vitro using methods described below.

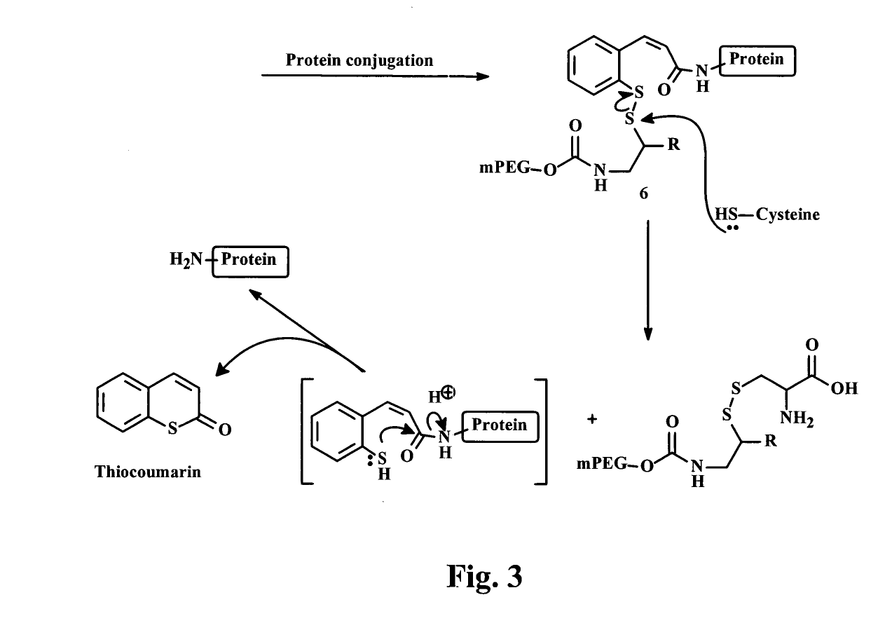

The cleavage reaction may produce two or three cleavage products initially, depending on the structure of the conjugate. For example, FIG. 3 shows the mechanism of thiolytic cleavage of the mPEG-DTC-(NH-ligand) conjugate of FIG. 1, in a three-fragment cleavage reaction. The disulfide group of the ortho-dithiocinnamyl moiety is cleaved thiolytically, e.g. in the presence of cysteine (as illustrated) or other naturally occurring reducing agents. An exogenous reducing agent can also be administered to artificially induce thiolytic conditions sufficient for cleavage and decomposition of the conjugate, or to accelerate cleavage.

As shown in FIG. 3, upon cleavage, the generated thiol group on the five- or six-membered ring (here a benzene ring) displaces the amine-containing ligand from the amide moiety, in a ring-closing reaction. The amine-containing compound is regenerated in its natural, unmodified form. R², or mPEG in this case, remains attached to L, which is now conjugated to the thiol-containing cleavage reagent, cysteine. The third entity generated, formed in the ring closing reaction, is the known, stable compound thiocoumarin, or a derivative thereof, depending on the substitution of the conjugate.

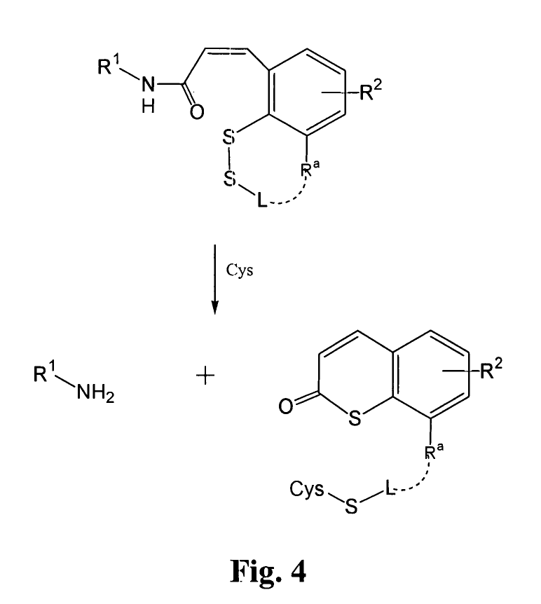

FIG. 4 illustrates cleavage of the conjugate in an embodiment in which L and R^{a} are linked, resulting in a two-fragment cleavage. In the embodiment of FIG. 1C, the conjugate is again structured on the dithiocinnamyl group. R² is attached to the aromatic ring, as in structure (iv) above. For example, R² could be PEG linked via a carbamate, as in FIG. 1.

Alternatively, R² could be attached to the L-R^{a} ring, as in structure (iii) above. Upon cleavage, the conjugated molecule is again released in its native form (e.g. R¹NH₂), via a similar mechanism. The second fragment is a thiocoumarin derivative attached to both the residue of the cleaving reagent (shown in FIG. 4 as cysteine) and to the polymer or other group R².

Thiolytic cleavage of a conjugate under biologically relevant conditions can be demonstrated by incubation with a physiologically present thiol, such as cysteine, glutathione, or albumin (Zalipsky et al., Proceed. Int'l. Symp. Control Rel. Bioact. Mater. 28:73 (2001)). Generation of the free protein or other released molecule can be monitored by SDS-PAGE. The rate of cleavage can be monitored by observing the concentration of the conjugate species as it disappears with time, or by measuring the free protein (or other released molecule) as it appears. Since thiocoumarin derivatives are generally reported to be chromophoric, the released thiocoumarin or derivative thereof can generally be easily detected as well. The rate of release of thiocoumarin can be observed by fluorescence spectroscopy.

If the conjugated molecule is biologically inactive in conjugated form, one can monitor cleavage by observing the restoration of biological activity (see e.g. Zalipsky et al., “Reversible PEGylation: thiolytic regeneration of active protein from its polymer conjugates”, in PEPTIDES:THE WAVE OF THE FUTURE, M. Lebl, R. A. Houghton, eds., Amer. Peptide Soc., 2001, p. 953; R. B. Greenwald et al., Bioconjugate Chem. 14:395 (2003)). The rate of thiolytic cleavage can be decreased significantly by increasing the size of the R group adjacent the disulfide linkage, e.g. from methyl to isopropyl, tert-butyl, etc.

The present conjugates provide the benefits of stability, when stored in the absence of a reducing agent, and cleavage at pharmaceutically useful rates in the presence of a suitable reducing agent, such as a thiol. Storage stability in particular is superior to that of the conjugates described in Greenwald et al., U.S. Pat. No. 6,214,340, which are based on cleavable phenyl esters. Such esters are subject to hydrolysis, generally at a greater rate than alkyl esters (see e.g. Quick et al., 1978; Blay et al., 1988; March 1992). Such hydrolysis could occur under ambient storage conditions, which is much less likely for reductive cleavage.

III. Exemplary Applications of the Subject Conjugates

A. Liposome Compositions Comprising an mPEG-Lipid Conjugate of the Invention

In one embodiment, the amine-containing ligand compound is an amine-containing lipid. Lipids, as referred to herein, intend water-insoluble molecules which typically have at least one hydrocarbon chain (“tail”) containing at least about eight carbon atoms, more preferably an acyl hydrocarbon chain containing between about 8-24 carbon atoms. A preferred lipid is a lipid having an amine-containing polar head group and an acyl chain. Exemplary lipids are phospholipids having a single acyl chain, such as stearoylamine, or two acyl chains. Preferred phospholipids with an amine-containing head group include phosphatidylethanolamine and phosphatidylserine. The lipid tail(s) preferably have between about 12 to about 24 carbon atoms and can be fully saturated or partially unsaturated. One preferred lipid is distearoylphosphatidylethanolamine (DSPE); however, those of skill in the art will appreciate the wide variety of lipids that fall within this description. It will also be appreciated that the lipid can naturally include an amine group or can be derivatized to include an amine group. Other lipid moieties that do not include a hydrocarbon chain as described above, e.g. cholesterolamine, are also suitable.

In one embodiment, the conjugates of the invention are formulated into liposomes. Liposomes are closed lipid vesicles used for a variety of therapeutic purposes, and in particular, for carrying therapeutic agents to a target region or cell by systemic administration. In particular, liposomes having a surface coating of hydrophilic polymer chains, such as polyethylene glycol (PEG), are desirable as drug carriers, since these liposomes offer an extended blood circulation lifetime over liposomes lacking the polymer coating. The polymer chains in the polymer coating shield the liposomes and form a “stiff brush” of water solvated polymer chains about the liposomes. Thus, the polymer acts as a barrier to blood proteins, preventing binding of the protein and recognition of the liposomes for uptake and removal by macrophages and other cells of the reticuloendothelial system.

Typically, liposomes having a surface coating of polymer chains are prepared by including in the lipid mixture between about 1 to about 20 mole percent of the lipid-polymer conjugate. The actual amount of lipid-polymer conjugate may be higher or lower, depending on the molecular weight of the polymer.

In various embodiments, the polymer chains in the above-referenced 1 to about 20 mole percent of lipids are attached to the lipids via the cleavable linking structures shown herein, or, in a preferred embodiment, by a combination of such linkages with linkages which are more stable in vivo. In this case, higher molecular weight polymer chains are preferably linked via the cleavable linking structures shown herein, and shorter polymer chains by more stable linkages.

In other embodiments, some or all of the polymer chains contain a targeting moiety, as noted above, at the free terminus.

Liposomes containing the polymer-lipid conjugate of the invention, preferably where R³ and/or R⁴ (in the definition of L for structure I) is non-hydrogen, have a blood circulation lifetime that is longer than liposomes containing polymer-lipid conjugates in which the polymer and lipid are joined by an aliphatic disulfide bond.

Importantly, cleavage of the polymer-lipid conjugates of the invention results in regeneration of the original lipid in unmodified form. This is desirable because unnatural, modified lipids can have undesirable in vivo effects. At the same time, the conjugate is stable when stored in the absence of reducing agents.

B. Polypeptide Conjugates

In another embodiment, the invention includes a conjugate as described above, where the amine-containing ligand compound is a polypeptide. In a preferred synthetic reaction scheme for preparation of a polymer-polypeptide conjugate of the invention, a mPEG-DTC-leaving group compound, such as shown at 5 in FIG. 2, is prepared according to a synthetic route such as that described in Examples 1-5. The leaving group may be, for example, N-hydroxy succinimide, as shown, nitrophenyl carbonate, or any one of the others described above. The R group adjacent the disulfide can be H, CH₃, C₂H₅ or the like and is selected according to the desired rate of disulfide cleavage. The mPEG-DTC-NHS compound 5, or equivalent, is then coupled to an amine moiety in a polypeptide to form a urethane (carbamate) linkage.

Attachment of polymer chains, such as PEG, to a polypeptide often diminishes the enzymatic or other biological activity, e.g., receptor binding, of the polypeptide. However, polymer modification of a polypeptide provides the benefit of increased blood circulation lifetime of the polypeptide. In the present invention, the polymer-polypeptide conjugate is administered to a subject. As the conjugate circulates, exposure to physiologic reducing conditions, such as blood cysteine and other in vivo thiols, initiates cleavage of the hydrophilic polymer chains from the polypeptide. As the polymer chains are released from the polypeptide, the biological activity of the polypeptide is gradually restored. In this way, the polypeptide initially has a sufficient blood circulation lifetime for biodistribution, and over time regains its full biological activity as the polymer chains are cleaved.

Some or all of the polymer chains may contain a targeting moiety, as noted above, at the free terminus.

In various embodiments, the polymer chains are attached to the polypeptide via the cleavable linking structures shown herein, or by a combination of such linkages with linkages which are more stable in vivo. The latter approach allows for attachment of PEG chains to amino groups in the polypeptide essential for biological activity with a reversible linkage, and attachment to amino groups that are not essential to peptide activity with a more stable linkage.

It will be appreciated that any of the hydrophilic polymers described above are contemplated for use. In preferred embodiments, the polymer is a polyalkylene glycol, preferably polyethylene glycol (PEG). The molecular weight of the polymer is selected depending on the polypeptide, the number of reactive amines on the polypeptide, and the desired size of the polymer-modified conjugate.

Polypeptides contemplated for use are unlimited and can be naturally-occurring or recombinantly produced polypeptides. Small, human recombinant polypeptides are preferred, and polypeptides in the range of 10-30 KDa are preferred. Exemplary polypeptides include cytokines, such as tumor necrosis factor (TNF), interleukins and interferons, erythropoietin (EPO), granulocyte colony stimulating factor (GCSF), enzymes, and the like. Viral polypeptides are also contemplated, where the surface of a virus is modified to include one or more polymer chain linked via a cleavable linkage as described herein. Modification of a virus containing a gene for cell transfection would extend the circulation time of the virus and reduce its immunogenicity, thereby improving delivery of an exogenous gene.

C. Amine-Containing Drug Conjugates

In yet another embodiment of the invention, the amine-containing ligand of structure I above is derived from an amine-containing drug. Modification of therapeutic drugs with PEG, for example, is effective to improve the blood circulation lifetime of the drug and to reduce any immunogenicity.

The conjugate is prepared according to any of the reaction schemes described above, with modifications as necessary to provide for the particular drug. A wide variety of therapeutic drugs have a reactive amine moiety, and the invention contemplates any such drugs with no limitation. Examples include mitomycin C, bleomycin, doxorubicin and ciprofloxacin.

### EXAMPLES

The following examples further illustrate the invention described herein and are in no way intended to limit the scope of the invention.

### Example 1

### Preparation of cis-mercaptocinnamic Acid (2) (See FIG. 2)

This compound can be synthesized according to the procedure published by J. A. Panetta and H. Rapoport (J. Org. Chem. 47:2626-2628 (1982)), as described below.

A solution of (2.0 g, 18 mmol) of freshly distilled thiophenol (1; see FIG. 2), triethyl orthopropiolate (prepared according to the procedure of H. Stetter et al., Synthesis 207 (1973)) (3.27 g, 19 mmol) and pivalic acid (1.6 g, 15 mmol) in 10 ml of p-cymene was heated at reflux for 26 h. The solvent was removed, and the residue was chromatographed on silica gel, using hexane/ether (9/1) as eluent, to give 2.79 g (75% yield) of ethyl 2-mercaptocinnamate as a pale yellow liquid: IR 3000, 1720, 1600, 1495, 1440 cm⁻¹; NMR δ 7.65 (d, 1H), 7.1-7.45(m, 4H), 5.55(d, 1H), 4.05(q, 2H), 1.15(t, 3H); MS calculated for C₁₁H₁₂O₂S m/e 208.0558 (M+), found 208.0560.

The above synthesized ethyl 2-mercaptocinnamate (1 g, 5.4 mmol) was dissolved in 10 ml of 95% ethanol, and KOH (0.75 g, 13.4 mmol) was added. The reaction mixture was heated at reflux for 2 h, then cooled to 25° C. and acidified with 5% aqueous HCl. The aqueous phase was extracted with ether (3×20 ml), and the combined organic fractions were washed with water (20 ml) and brine (20 ml), dried over anhydrous sodium sulfate, and concentrated to give 0.85 g (87%) of crystalline cinnamic acid 2. M.p. 128-129° C.; UV λₘₐₓ=250 nm (ε=7350 M⁻¹ cm⁻¹), and 275 (9700); NMR δ 7.8 (d, 1H), 7.15-7.45(m, 4H), 5.5(d, 1H); Anal. Calculated for C₉H₈O₂S: C, 60.0; H, 4.5 Found: C, 60.2; H, 4.6.

### Example 2

### Preparation of 2-mercaptopropylamine Hydrochloride (3, R═CH₃)

This compound can be prepared from the corresponding amino alcohol, e.g. in accordance with the procedure described by T. C. Owen, J. Chem. Soc. C 1373-1376 (1967). Briefly, the compound is esterified with sulfuric acid to the aminoalkyl sulfate, followed by cyclization with carbon disulfide and alkali to the thiazolidinethione, which is then hydrolyzed to give the product. Analogous aminoalkanethiol derivatives, having various R substituents, can be prepared in a similar fashion.

Formation of the Dithiocinnamic Acid (DTC) Linker:

### Example 3

### Synthesis of Mixed Disulfide 2-aminopropyl-dithiocinnamic Acid (4)

Reaction of 2-mercaptopropylamine hydrochloride (3) (Example 2) with diethyl azidocarboxylate, followed by reaction with cis-mercaptocinnamic acid (2) (Example 1), in accordance with the procedure described by T. Mukaiyama et al., Tetrahedron Letters 56:5907-5908 (1968) provides the mixed disulfide (4). Alternatively, (3) can be reacted with methoxycarbonylsulfenyl chloride to form 2-(methoxycarbonyldithio)propylamine hydrochloride, as described in S. J. Brois et al., J. Am. Chem. Soc. 92:7629-31 (1970), followed by reaction with mercaptocinnamic acid (2) to form the mixed disulfide (4) (see S. Zalipsky et al., Bioconjugate Chem. 10:703-7 (1999)).

### Example 4

### Synthesis of mPEG-Urethane-Linked Dithiocinnamic Acid (mPEG-DTC, 5a)

This transformation can be accomplished by reaction of 2-aminopropyl disulfanylcinnamic acid (4) (Example 3) with mPEG-chloroformate. See for example, S. Zalipsky and S. Menon-Rudolph in Poly(ethylene glycol): Chemistry and Biological Applications, J. M. Harris & S. Zalipsky, eds., Amer. Chem. Soc., Washington, D.C., 1997 pp. 318-341. The mPEG chloroformate is easily generated by phosgenation of an anhydrous mPEG-OH solution, according to S. Zalipsky et al., Biotechnol. Appl. Biochem. 15:100-114 (1992).

Alternatively, the urethane linkage can be formed by reaction of 2-aminopropyl disulfanylcinnamic acid (4) (Example 3) with mPEG-succinimidyl carbonate, according to the procedure of H.-C. Chiu et al., Bioconjugate Chem. 4:290-295 (1993); Zalipsky et al., (1992), cited above; or Zalipsky et al., (1997), cited above.

### Example 5

### Synthesis of mPEG-DTC NHS Ester (5)

mPEG-urethane-linked dithiocinnamic acid (5a) can be converted to its N-hydroxy succinimide ester using esterification procedures known in the art, e.g. as described in G. W. Anderson et al., J. Amer. Chem. Soc. 86:1839 (1964); R. Knorr et al., Tetrahedron Lett. 30:1927 (1989); or M. Wilchek et al., Bioconjugate Chem. 5:491 (1994).

### Example 6

### Preparation of mPEG-DTC-Protein Conjugates (6)

The N-hydroxysuccinimide ester (5) can be reacted with an amino group of a protein, typically in an aqueous buffer at neutral or basic pH (pH 7-9), according to various published procedures. For representative procedures, see e.g. S. Zalipsky et al., (1992), cited above; H. C. Chiu et al., Bioconjugate Chem. 4:290 (1993). Depending on various reaction parameters, such as the ratio of the mPEG reagent and amino groups on the protein, pH of the reaction buffer, temperature, and duration of the reaction, one can obtain a range of conjugate species with varying degrees of PEGylation. Conjugate mixtures of the general formula (mPEG)ₙ-protein can be fractionated by various chromatographic techniques. It is often possible to purify (mPEG)ₙ-protein conjugates with n=1, e.g. by ion-exchange chromatography.

### Example 7

### Thiolytic Cleavage of mPEG-DTC-Protein Conjugates

De-PEGylation in response to thiolysis under biologically relevant conditions can be demonstrated by incubation of the conjugate with a physiologically present thiol, such as cysteine, glutathione, or albumin (S. Zalipsky et al., Proceed. Int'l. Symp. Control Rel. Bioact. Mater. 28:73 (2001). Conversion of the cleavable PEG-protein conjugates to the free protein can be monitored, for example, by SDS-PAGE. The rate of the reaction can be followed by measuring the concentration of the conjugate species as its disappear with time, or by measuring the free protein as it appears. If the conjugate is devoid of biological activity as the result of PEGylation, one can measure the time course of the restoration of biological activity of the protein under the cleavage conditions (S. Zalipsky et al., Reversible PEGylation: thiolytic regeneration of active protein from its polymer conjugates, in Peptides: The Wave of the Future, M. Lebl and R. A. Houghton, eds., Amer. Peptide Soc., 2001, p. 953; R. B. Greenwald et al., Bioconjugate Chem. 14:395 (2003).

Although the invention has been described with respect to particular embodiments, it will be apparent to those skilled in the art that various changes and modifications can be made without departing from the invention.

1. A conjugate having the structure I: 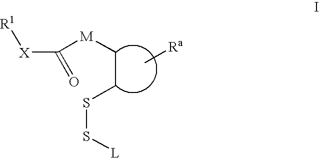
wherein
R¹X is an amine- or hydroxyl-containing ligand, such that X is oxygen, primary nitrogen or secondary nitrogen;
M is selected from cis —CR^{b}═CR^{c}—, —CR^{b}R^{d}—, and —CR^{b}R^{d}—CR^{c}R^{e}—, wherein each of R^{b}, R^{c} R^{d}, and R^{e} is independently selected from H, methyl, substituted methyl, fluoro, and chloro, where methyl may be substituted with hydroxyl, fluoro, or chloro;
the D-shaped structure represents a five- or six-membered ring to which M and the disulfide group S—S are attached in a cis-1,2- or ortho orientation;
R^{a} represents hydrogen or one or more substituents on the ring selected from R, OR, C(O)OH, C(O)OR, OC(O)OR, C(O)NR₂, OC(O)NR₂, cyano, nitro, halogen, and a further fused ring, where R is C₁-C₆ hydrocarbyl, which may be further substituted with halogen; and
L is a linear or branched C₁-C₆ alkyl group, which may be further substituted with aryl or aralkyl;
wherein L and R^{a} may together form a ring;
and wherein the conjugate further comprises, attached to L, to R^{a}, or to the five- or six-membered ring, a lipid or a hydrophilic polymer.

2. The conjugate of claim 1, wherein L and R^{a} do not form a ring.

3. The conjugate of claim 2, comprising a hydrophilic polymer attached to L or to R^{a}.

4. The conjugate of claim 2, wherein the five- or six-membered ring is an aromatic ring.

5. The conjugate of claim 4, wherein the aromatic ring is a benzene ring, and M is cis —CR^{b}═CR^{c}—, such that the conjugate has the structure Ia:

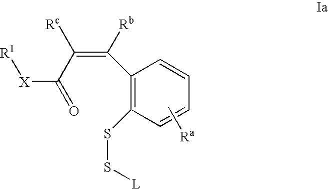

6. The conjugate of claim 5, wherein each of R^{b} and R^{c} is hydrogen.

7. The conjugate of claim 6, comprising a hydrophilic polymer attached to L and not to R^{a}.

8. The conjugate of claim 7, wherein R^{a} is hydrogen.

9. The conjugate of claim 5, wherein L has the structure —CR³R⁴—CR⁵R⁶—, such that —CR³R⁴ is attached to the disulfide group, where R³ and R⁴ are independently selected from H, alkyl, aryl, and aralkyl, and R⁵ and R⁶ are independently selected from H and methyl.

10. The conjugate of claim 9, wherein each of R³ and R⁴ is independently selected from hydrogen, methyl, ethyl, and propyl.

11. The conjugate of claim 10, wherein R⁴ is H and R³ is selected from the group consisting of hydrogen, methyl, ethyl, and propyl.

12. The conjugate of claim 1, wherein L and R^{a} are attached to the five- or six-membered ring in a cis-1,2- or ortho orientation, and L and R^{a} together form a further five- to seven-membered ring.

13. The conjugate of claim 12, comprising a hydrophilic polymer attached to the five- or six-membered ring.

14. The conjugate of claim 12, comprising a hydrophilic polymer attached to said further five- to seven-membered ring.

15. A method for administering an amine- or hydroxyl-containing molecule R²XH to the bloodstream, comprising:
administering to the bloodstream a conjugate having the structure: 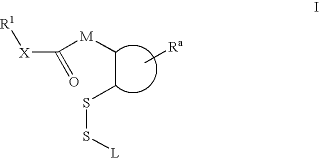
wherein
R¹X is an amine- or hydroxyl-containing ligand, such that X is oxygen, primary nitrogen or secondary nitrogen;
M is selected from cis —CR^{b}═CR^{c}—, —CR^{b}R^{d}—, and —CR^{b}R^{d}—CR^{c}R^{e}—, wherein each of R^{b}, R^{c} R^{d}, and R^{e} is independently selected from H, methyl, substituted methyl, fluoro, and chloro, where methyl may be substituted with hydroxyl, fluoro, or chloro;
the D-shaped structure represents a five- or six-membered ring to which M and the disulfide group S—S are attached in a cis-1,2- or ortho orientation;
R^{a} represents hydrogen or one or more substituents on the ring selected from R, OR, C(O)OH, C(O)OR, OC(O)OR, C(O)NR₂, OC(O)NR₂, cyano, nitro, halogen, and a further fused ring, where R is C₁-C₆ hydrocarbyl, which may be further substituted with halogen; and
L is a linear or branched C₁-C₆ alkyl group, which may be further substituted with aryl or aralkyl;
wherein L and R^{a} may together form a ring;
and wherein the conjugate further comprises, attached to L, to R^{a}, or to the five- or six-membered ring, a lipid or a hydrophilic polymer;
whereby said molecule R²XH is released from said conjugate via an in vivo thiolytic cleavage reaction of said conjugate.

16. The method of claim 15, wherein L and R^{a} do not form a ring.

17. The method of claim 16, wherein a hydrophilic polymer is attached to L or to R^{a}.

18. The method of claim 16, wherein the five- or six-membered ring is a benzene ring, and M is cis —CR^{b}═CR^{c}—, such that the conjugate has the structure Ia:

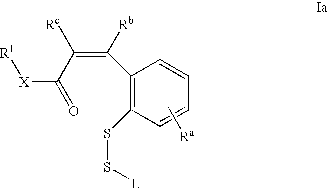

19. The method of claim 18, wherein a hydrophilic polymer is attached to L and not to R^{a}.

20. The method of claim 19, wherein R^{a} is hydrogen.

21. The method of claim 18, wherein L has the structure —CR³R⁴—CR⁵R⁶—, such that —CR³R⁴ is attached to the disulfide group, where R³ and R⁴ are independently selected from H, alkyl, aryl, and aralkyl, and R⁵ and R⁶ are independently selected from H and methyl.

22. The method of claim 21, wherein each of R³ and R⁴ is independently selected from hydrogen, methyl, ethyl, and propyl.

23. The method of claim 15, wherein L and R^{a} are attached to the five- or six-membered ring in a cis-1,2- or ortho orientation, and L and R^{a} together form a further five- to seven-membered ring.

24. The method of claim 23, wherein a hydrophilic polymer is attached to the five- or six-membered ring.

25. The method of claim 23, wherein a hydrophilic polymer is attached to said further five- to seven-membered ring.

26. The method of claim 15, further comprising monitoring the release of said molecule via detection of a fluorescent moiety released by said cleavage reaction.

27. A liposome having a surface coating of hydrophilic polymer chains, and comprising a lipid-polymer conjugate having the structure I: 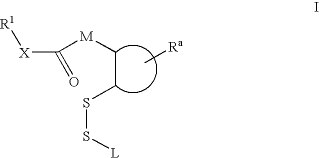
wherein
R¹X is an amine- or hydroxyl-containing lipid, such that X is oxygen, primary nitrogen or secondary nitrogen;
M is selected from cis —CR^{b}═CR^{c}—, —CR^{b}R^{d}—, and —CR^{b}R^{d}—CR^{c}R^{e}—, wherein each of R^{b}, R^{c} R^{d}, and R^{e} is independently selected from H, methyl, substituted methyl, fluoro, and chloro, where methyl may be substituted with hydroxyl, fluoro, or chloro;
the D-shaped structure represents a five- or six-membered ring to which M and the disulfide group S—S are attached in a cis-1,2- or ortho orientation;
R^{a} represents hydrogen or one or more substituents on the ring selected from R, OR, C(O)OH, C(O)OR, OC(O)OR, C(O)NR₂, OC(O)NR₂, cyano, nitro, halogen, and a further fused ring, where R is C₁-C₆ hydrocarbyl, which may be further substituted with halogen; and
L is a linear or branched C₁-C₆ alkyl group, which may be further substituted with aryl or aralkyl;
wherein L and R^{a} may together form a ring;
and wherein the conjugate comprises, attached to L, to R^{a}, or to the five- or six-membered ring, a hydrophilic polymer.

28. The liposome of claim 27, wherein L and R^{a} do not form a ring, and a hydrophilic polymer is attached to L.

29. The liposome of claim 27, wherein the five- or six-membered ring is a benzene ring, and M is cis —CR^{b}═CR^{c}—, such that the conjugate has the structure Ia:

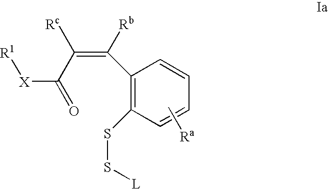

30. The liposome of claim 29, wherein R^{a} is hydrogen.

31. The liposome of claim 29, wherein L has the structure —CR³R⁴—CR⁵R⁶—, such that —CR³R⁴ is attached to the disulfide group, where R³ and R⁴ are independently selected from H, alkyl, aryl, and aralkyl, and R⁵ and R⁶ are independently selected from H and methyl.

32. The liposome of claim 31, wherein each of R³ and R⁴ is independently selected from hydrogen, methyl, ethyl, and propyl.

33. The liposome of claim 26, wherein L and R^{a} are attached to the five- or six-membered ring in a cis-1,2- or ortho orientation, and L and R^{a} together form a further five- to seven-membered ring.

34. The liposome of claim 33, wherein a hydrophilic polymer is attached to the five- or six-membered ring or to said further five- to seven-membered ring.

35. The liposome of claim 27, further comprising an entrapped therapeutic agent.

36. A conjugate obtainable by reaction of an amine- or hydroxyl-containing molecule with a compound having the structure II: 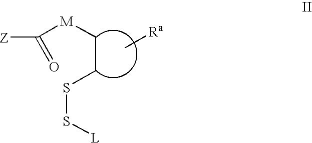
wherein
Z is a leaving group displaceable by a hydroxyl or amino group;
M is selected from cis —CR^{b}═CR^{c}—, —CR^{b}R^{d}—, and —CR^{b}R^{d}—CR^{c}R^{e}—, wherein each of R^{b}, R^{c}, R^{d}, and R^{e} is independently selected from H, methyl, substituted methyl, fluoro, and chloro, where methyl may be substituted with hydroxyl, fluoro, or chloro;
the D-shaped structure represents a five- or six-membered ring to which M and the disulfide group S—S are attached in a cis-1,2- or ortho orientation;
R^{a} represents hydrogen or one or more substituents on the ring selected from R, OR, C(O)OH, C(O)OR, OC(O)OR, C(O)NR₂, OC(O)NR₂, cyano, nitro, halogen, and a further fused ring, where R is C₁-C₆ hydrocarbyl, which may be further substituted with halogen; and
L is a linear or branched C₁-C₆ alkyl group, which may be further substituted with aryl or aralkyl;
wherein L and R^{a} may together form a ring;
and wherein the compound further comprises, attached to L, to R^{a}, or to the five- or six-membered ring, a lipid or a hydrophilic polymer.

37. The conjugate of claim 36, wherein L and R^{a} do not form a ring.

38. The conjugate of claim 37, comprising a hydrophilic polymer attached to L.

39. The conjugate of claim 38, wherein the five- or six-membered ring is a benzene ring, and M is cis —CR^{b}═CR_{c}—, such that the compound has the structure IIa:

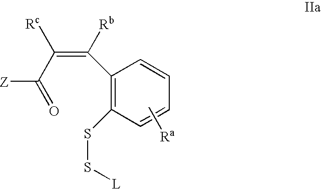

40. The conjugate of claim 39, wherein L has the structure —CR³R⁴—CR⁵R⁶—, such that —CR³R⁴ is attached to the disulfide group, where R³ and R⁴ are independently selected from H, alkyl, aryl, and aralkyl, and R⁵ and R⁶ are independently selected from H and methyl.
